(19) United States
(12) Patent Application Publication
(10) Pub. No.: US 2024/0318148 A1
(43) Pub. Date: Sep. 26, 2024

(54) Fluorescently Labeled Lentiviral Vector, And Preparation Methods And Use Thereof

(71) Applicant: UNION HOSPITAL TONGJI MEDICAL COLLEGE HUST, Wuhan (CN)

(72) Inventors: Heng MEI, Wuhan (CN); Yu HU, Wuhan (CN); Zhaozhao CHEN, Wuhan (CN)

(21) Appl. No.: 18/527,746

(22) Filed: Dec. 4, 2023

(30) Foreign Application Priority Data
Mar. 24, 2023 (CN) 202310295684.1

(51) Int. Cl. C12N 7/00; C09B 23/06; C12N 5/00; C12N 5/071; C12N 15/85

(52) U.S. Cl. CPC C12N 7/00; C09B 23/06; C12N 5/0037; C12N 5/0686; C12N 15/85; C12N 2500/34; C12N 2740/15043; C12N 2740/15051

(57) ABSTRACT

The present disclosure belongs to lentiviral labeling technology, and in particular relates to a fluorescently labeled lentiviral vector, and a preparation method and use thereof. The preparation method of a fluorescently labeled lentiviral vector includes: preparation of N₃-LVs: subjecting a 293T cell and an azido sugar to co-incubation, such that a cell membrane system is azidated through sugar metabolism to obtain an N₃-293T cell capable of expressing an azido group on an envelope surface, and subjecting the N₃-293T cell and a plasmid packaging system to co-transfection to form a lentiviral vector N₃-LVs; and preparation of Cy5-LVs: mixing the N₃-LVs with a sufficient amount of an azide dibenzocyclooctyne-cyanine 5 (DBCO-Cy5) to allow co-incubation, removing excess DBCO-Cy5, and collecting a virus concentrate to obtain a fluorescently labeled lentiviral vector Cy5-LVs. In the present disclosure, a simple and easy lentiviral fluorescent labeling technology is provided by covalently modifying fluorescein on an envelope surface of a lentivirus based on bioorthogonal click chemistry with a low cost.

### TECHNICAL FIELD

The present disclosure belongs to lentiviral labeling technology, and in particular relates to a fluorescently labeled lentiviral vector, and a preparation method and use thereof.

### BACKGROUND

Lentivirus can effectively integrate foreign genes (or RNAi) into a host cell genome, and then stably and persistently express a target protein or mediate gene silencing. Lentiviral vectors are widely used in gene therapy, cell therapy, gene editing, transgenic animals, drug research, and construction of stably transduced cell lines. The lentivirus infects a host cell by binding an envelope protein to a surface receptor on the host cell, fuses with a cell membrane to enter the host cell, and then integrates a target gene into a chromosome of the host cell through a series of biological processes. Fluorescent protein labels are generally used to detect whether lentiviral transfection is successful, but it is not possible to continuously and dynamically observe the real-time infection behavior of lentivirus in host cells. To date, there are no effective technical means to monitor and decipher a biological invasion process of the lentivirus.

### SUMMARY

In view of the above problems, the present disclosure provides a fluorescently labeled lentiviral vector, and a preparation method and use thereof. The present disclosure is mainly intended to solve the problems that the existing technology cannot achieve continuous and dynamic observation of the real-time infection behavior of lentivirus in host cells, making it impossible to conduct researches on the biological invasion process of lentivirus.

To solve the mentioned technical problems, the present disclosure adopts the following technical solution.

A first aspect of the present disclosure relates to a preparation method of a fluorescently labeled lentiviral vector, including the following steps:
preparation of N₃-LVs: subjecting a 293T cell and an azido sugar to co-incubation, such that a cell membrane system is azidated through sugar metabolism to obtain an N₃-293T cell capable of expressing an azido group on an envelope surface, and subjecting the N₃-293T cell and a plasmid packaging system to co-transfection to form a lentiviral vector N₃-LVs; and
preparation of Cy5-LVs: mixing the N₃-LVs with a sufficient amount of an azide dibenzocyclooctyne-cyanine 5 (DBCO-Cy5) to allow co-incubation, removing excess DBCO-Cy5, and collecting a virus concentrate to obtain a fluorescently labeled lentiviral vector Cy5-LVs.

A process of the mixing can be conventionally selected according to actual needs, such as stirring, ultrasonic oscillation and the like; a process of removing the excess DBCO-Cy5 can also be conventionally selected as needed, with a purpose of removing excess reactants. Specifically, centrifugal impurity removal can be adopted. The “sufficient amount of DBCO-Cy5” generally means an excess of reactants, with a purpose of fully reacting the N₃-LVs and completely occupying N₃ groups.

In some cases, the plasmid packaging system is selected from the group consisting of a three-plasmid packaging system and a four-plasmid packaging system;
the three-plasmid packaging system includes: a vector plasmid, and packaging plasmids PsPAX2, PMD2.G; and
the four-plasmid packaging system includes: the vector plasmid, and packaging plasmids pVSV-G, pGap/pol, and pRev;

The PsPAX2, PMD2.G, pVSV-G, pGap/pol, and pRev are all packaging plasmids, while the vector plasmid can be GFP.

In some cases, the N₃-LVs is prepared specifically according to the following steps:
subjecting the 293T cell and the azido sugar to the co-incubation and subculture in a cell medium, discarding the cell medium, and transferring a resulting culture product into a serum-free medium; and adding a mixture of a solution A and a solution B dropwise into the serum-free medium, replacing the serum-free medium with a Dulbecco's modified eagle medium (DMEM) complete medium containing fetal bovine serum, conducting transfection, aspirating a resulting cell culture supernatant to obtain a virus concentrate, thereby obtaining the N₃-LVs; a process of obtaining the virus concentrate may include: removing cell debris from the transfected cell culture supernatant, and conducting centrifugal filtration to obtain the virus concentrate; and the solution A is prepared by placing the vector plasmid, the PsPAX2, and the PMD2.G in a serum-reduced medium, and the solution B is prepared by dissolving polyethylenimine (PEI) in the serum-reduced medium; the solutions A and B can be prepared in advance or when being used; for example, the solution A is prepared from 18 μg of the GFP plasmid (vector plasmid), the PsPAX2, and the PMD2.G in a ratio of 9:6:3 in 250 μL of Opti-MEM, and the solution B is prepared from dissolving 45 μL of the PEI (1 μg/μL) in 250 μL of the Opti-MEM, and then mixing by pipetting; when being used in other application environments, a ratio of the solutions A to B can also be referred to the above range, and concentrations of the raw materials can be adjusted within an allowable range. When the four-plasmid packaging system is used, the vector plasmid, the pVSV-G, the pGap/pol, and the pRev are at a ratio of 10.5:1.5:5:3.

In some cases, the vector plasmid, the PsPAX2, and the PMD2.G in the solution A are at a ratio of 3:2:1 (for example, when the above three have a total volume of 18 μg, 9 μg, 6 μg, and 3 μg are used, respectively); and the solution B has the PEI at a concentration of 1 μg/μL.

In some cases, the 293T cell and the azido sugar subjected to the co-incubation and the subculture in the cell medium have a fusion degree of 80% to 90%. This fusion degree is an optimal situation; when the fusion degree is lower than the above range, as long as it does not affect the preparation of the N₃-LVs, any range should be regarded as falling within the protection scope of the present disclosure.

In some cases, the azido sugar is any one selected from the group consisting of N-azidoacetylmannosamine-tetraacylated (Ac4ManNAZ), N-azidoacetylgalactosamine-tetraacylated (Ac4GlaNAZ), and N-azidoacetylglucosamine-tetraacylated (Ac4GlcNAZ); in some cases, the Ac4ManNAZ has a concentration of 50 μM.

The 293T cell and the azido sugar are subjected to the co-incubation in the cell medium at 35° C. to 38° C.; and the N₃-LVs and a sufficient amount of the azide DBCO-Cy5 serving as a reaction probe are subjected to the co-incubation at 35° C. to 38° C. Without affecting an incubation effect, other temperatures should be equivalent to this protocol.

A second aspect of the present disclosure relates to a fluorescently labeled lentiviral vector N₃-LVs prepared by the preparation method of a fluorescently labeled lentiviral vector. Although the N₃-LVs are prepared by the aforementioned preparation method, it is not limited to being prepared by the aforementioned preparation method. As long as a corresponding lentiviral vector has the same properties as that involved in this application, any lentiviral vector should be considered to be within the protection scope of the present disclosure. Of course, the N₃-LVs mentioned in this application may be prepared by different and similar protocols. For example, changes in unstable conditions (such as concentration, temperature and so on) during the preparation cause differences in the performances of the N₃-LVs finally obtained, but these N₃-LVs products with slight differences in properties are all within the protection scope of the present disclosure.

A third aspect of the present disclosure relates to use of the N₃-LVs in preparation of a biological invasion model for a lentivirus. One of the main functions of this biological invasion model for a lentivirus is to study the real-time infection behavior of lentivirus in host cells. The model can not only detect whether lentivirus transfection is successful, but also realize continuous dynamic monitoring of infection behavior, thereby providing a better research model for deciphering a biological invasion process of the lentivirus.

A fourth aspect of the present disclosure relates to use of Ac4ManNAZ or DBCO-Cy5 in preparation of a fluorescently labeled lentiviral vector. During the preparation of fluorescently labeled lentiviral vector, the Ac4ManNAZ enables the cell membrane system to achieve azidation, and an envelope surface of the packaged lentivirus can also express azido groups. In this way, the lentiviral vector and the fluorescent probe DBCO-Cy5 can be coupled through a click reaction under mild conditions and without any catalyst, to achieve fluorescent labeling of the lentiviral vector.

The present disclosure has the following beneficial effects: a simple and easy lentiviral fluorescent labeling technology is provided by covalently modifying fluorescein on an envelope surface of a lentivirus based on bioorthogonal click chemistry with a low cost. This technology is a non-genetic modification process without the insertion of additional tag sequences into the target gene. Based on this labeling technology, it is possible to dynamically monitor the behavioral changes of lentivirus-infected host cells, thus showing a great application potential for exploring a biological invasion mechanism of the lentivirus.

### BRIEF DESCRIPTION OF THE DRAWINGS

FIG. 1 shows a preparation flow chart of the Cy5-LVs;

FIG. 2 shows an optimal bioorthogonal azidation concentration that does not affect a cell activity selected by a CCK8 method;

FIG. 3 shows azido groups on a surface of the 293T cell detected by flow cytometry;

FIG. 4 shows subcellular localization, time dependence, and dose dependence of an azidated protein analyzed by Western blotting;

FIG. 5 shows distribution of the azido groups in cells and co-localization with a cell membrane observed by a confocal microscope; where (A) is the distribution of the azido groups in cells observed by the confocal microscope, and (B) is the co-localization of the azido groups and the cell membrane observed by the confocal microscope;

FIG. 6 shows presence of the azido groups on the surface of lentivirus detected by dot immunoblotting assay;

FIG. 7 shows co-localization of lentivirus surface labels and DBCO-Cy5 analyzed by the confocal microscope; and

FIG. 8 shows transmission electron microscopy (TEM) images of LVs and N₃-LVs.

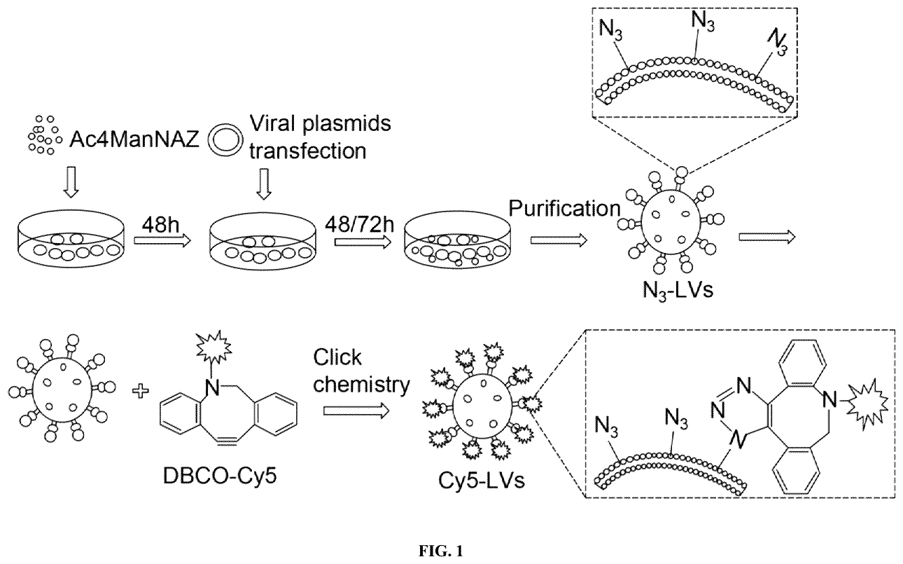

### DETAILED DESCRIPTION OF THE EMBODIMENTS

The present disclosure is further described below:

Screening of the optimal azido sugar and verification of the bioorthogonal click chemistry: three azido sugars, namely Ac4ManNAZ, Ac4GlaNAZ, and Ac4GlcNAZ were selected, a working concentration that did not affect cell activity was found out, and the most suitable azido sugar was selected for bioorthogonal reactions in the 293T cells.

a. Exploring the Optimal Working Concentration

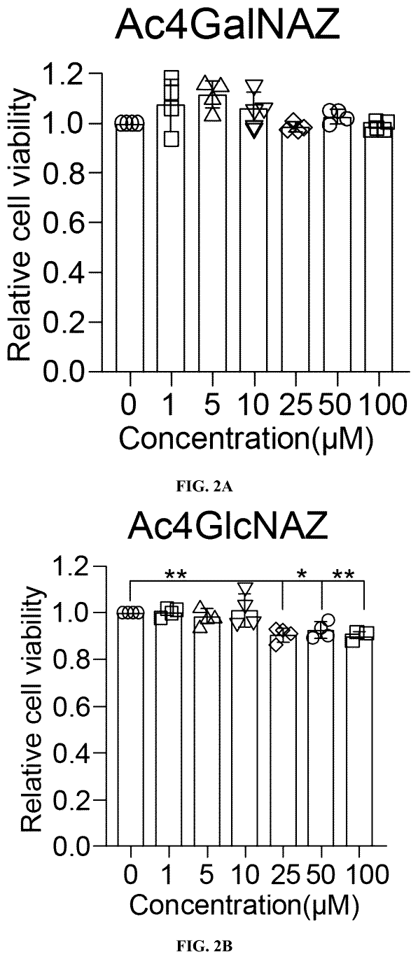

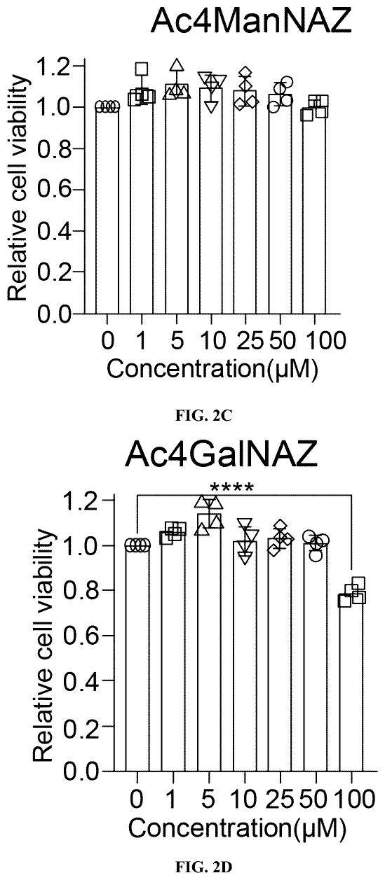

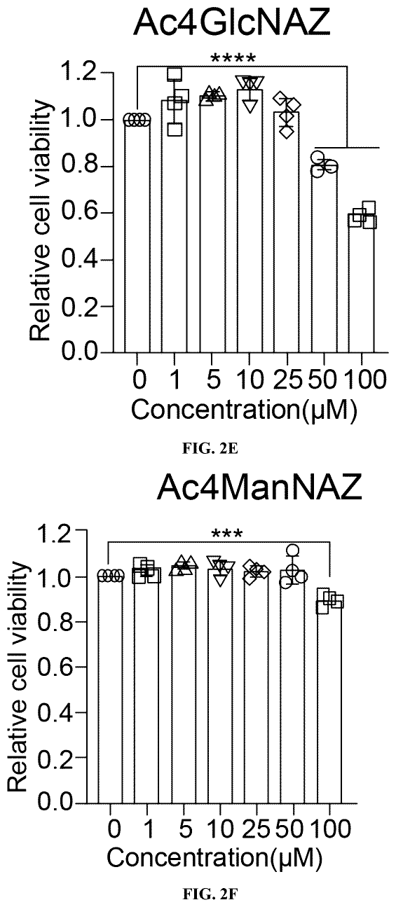

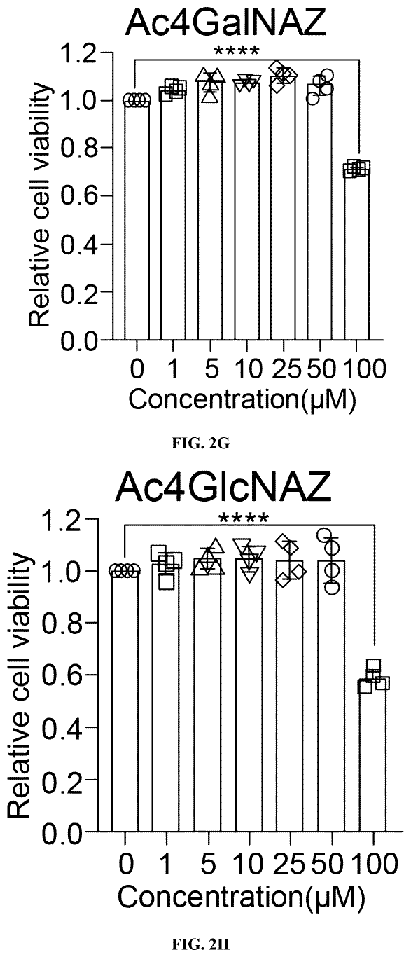

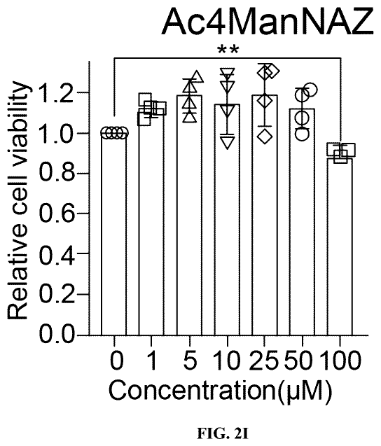

293T cells were pre-inoculated into a 96-well plate, with 1*10⁴ cells in each well, and then allowed to adhere well overnight. Ac4ManNAZ, Ac4GlaNAZ, and Ac4GlcNAZ solutions with gradient concentrations (0 μM, 1 μM, 5 μM, 10 μM, 25 μM, and 50 μM) were prepared, respectively, and the cells were added with above drugs and then incubated for 24 h, 48 h, and 72 h, respectively. The cytotoxicity of azido sugar to 293T cells was detected by a CCK8 method at various time points, and the results were shown in FIG. 2. The CCK8 experiments and flow cytometry showed that cells at a concentration of 50 μM had better viability and high bioorthogonal efficiency.

b. Detecting Azidation in 293T Cells by Flow Cytometry

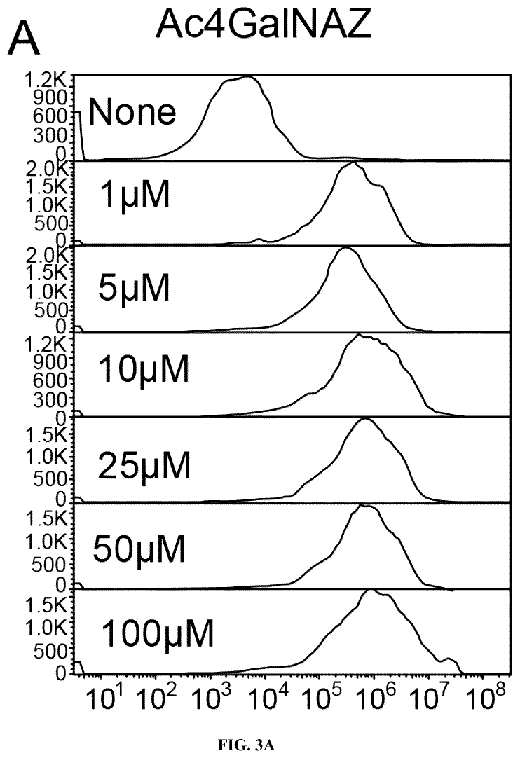

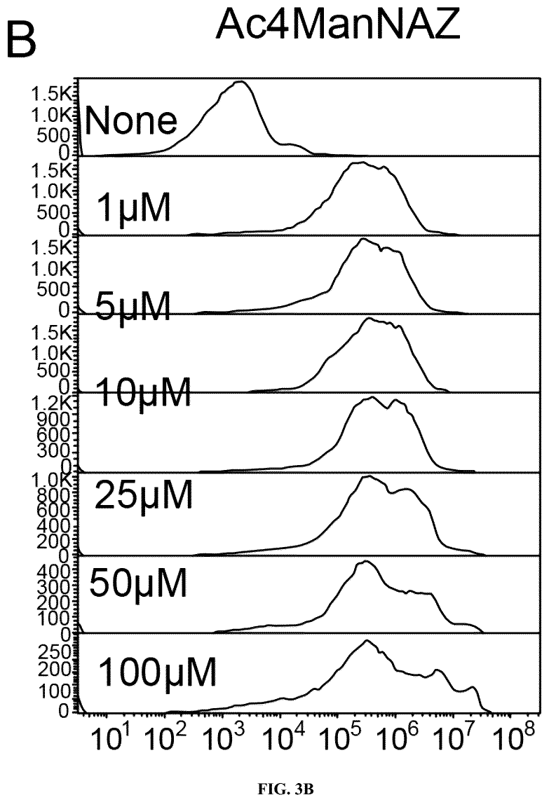

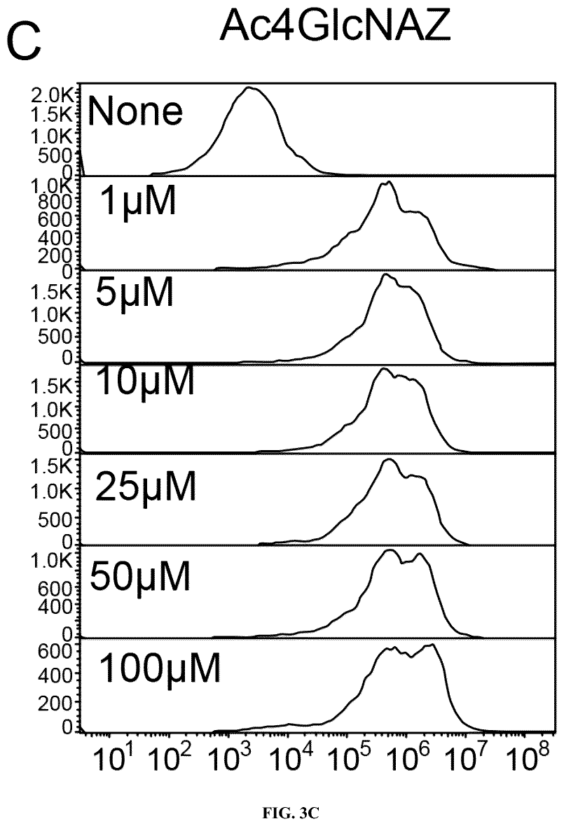

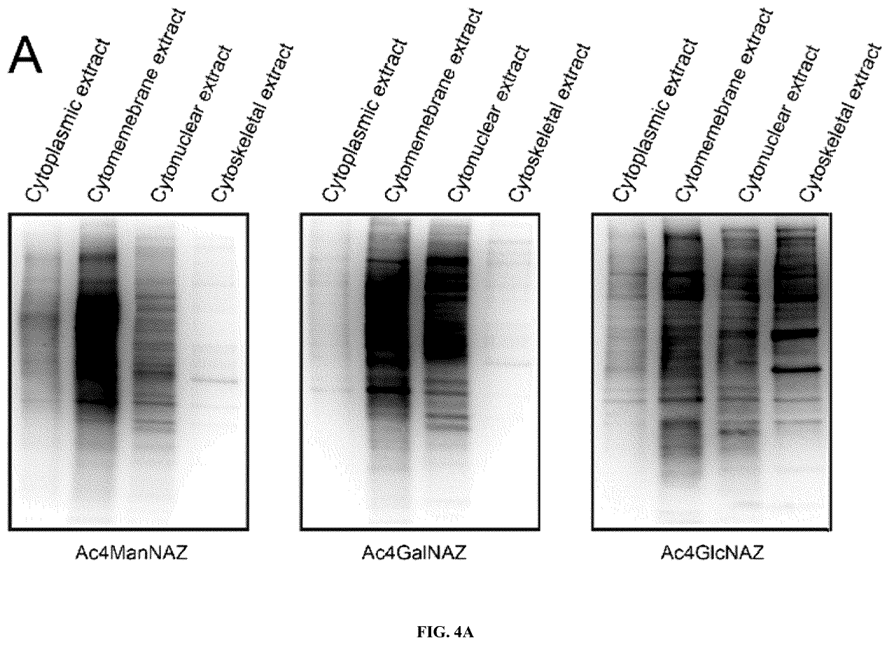

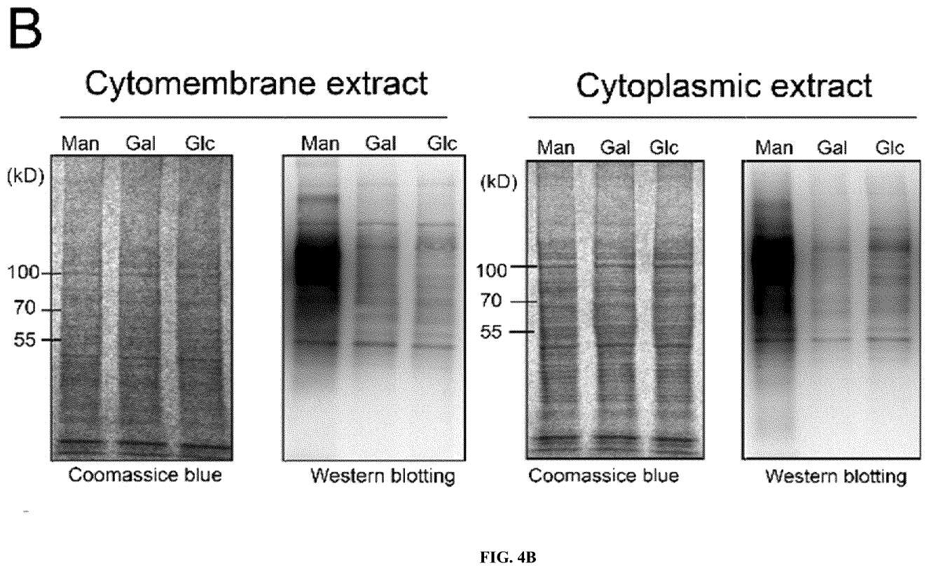

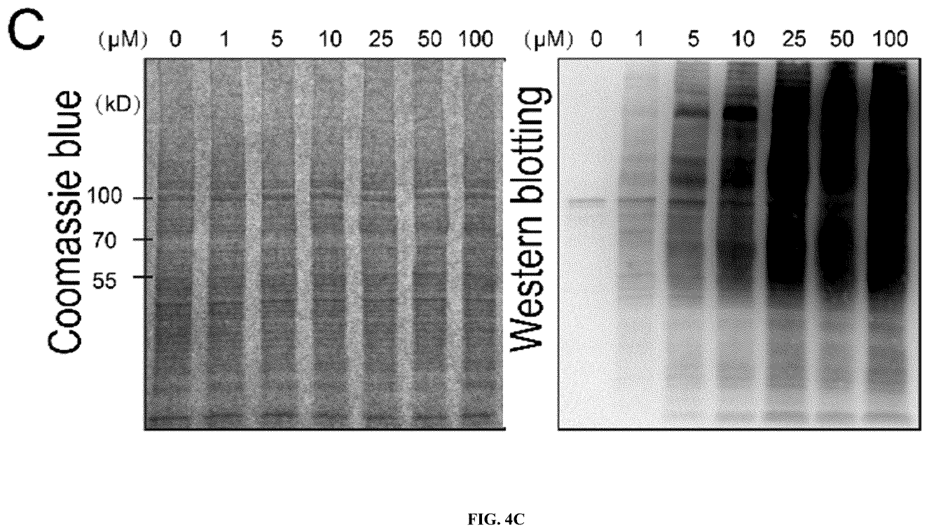

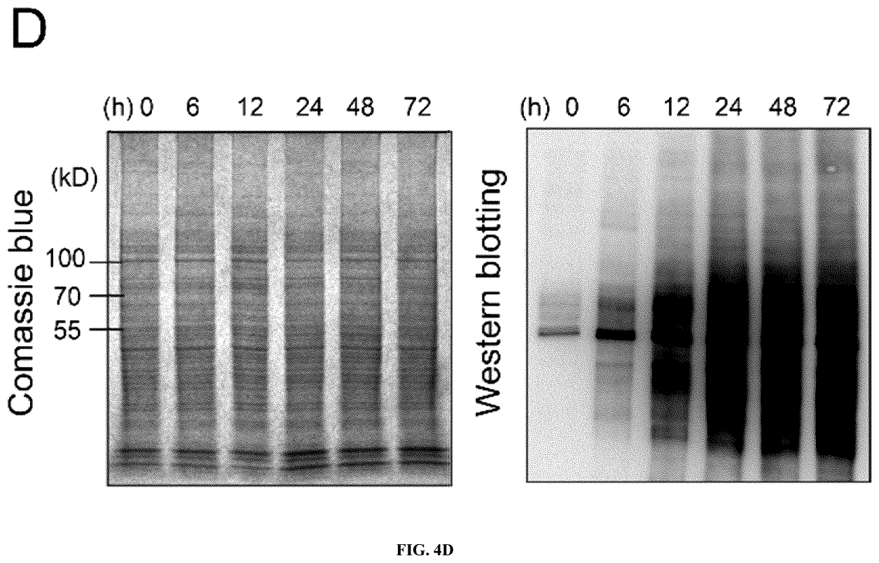

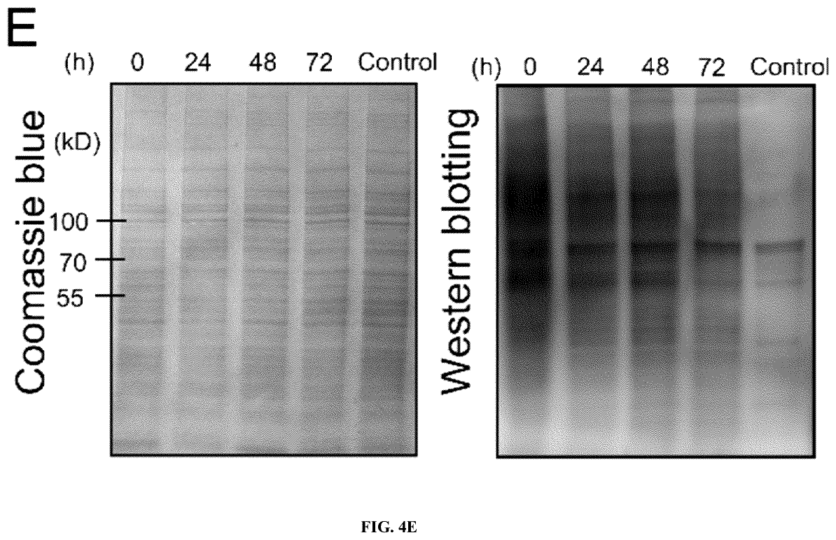

293T cells were pre-inoculated into a 6-well plate, with 1*10⁵ cells in each well, and then allowed to adhere well overnight. Ac4ManNAZ, Ac4GlaNAZ, and Ac4GlcNAZ solutions with gradient concentrations (0 μM, 1 μM, 5 μM, 10 μM, 25 μM, and 50 μM) were prepared, respectively, and the cells were added with above drugs and then incubated for 48 h separately. 10 μM of DBCO-Cy5 was added, the cells were incubated in a 37° C. incubator for 30 min, washed 3 times with PBS, and then fixated with 4% paraformaldehyde at room temperature for 15 min, and finally detected by flow cytometry. The results were shown in FIG. 3.

c. Colocalization of Azido Groups and Cell Membrane Analyzed by Laser Confocal Experiment

293T cells were pre-inoculated into a confocal dish, with 1*10⁵ cells in each well, and then allowed to adhere well overnight. 50 μm each of Ac4ManNAZ, Ac4GlaNAZ, and Ac4GlcNAZ solutions were prepared separately, and the cells were added with above drugs and then incubated for 48 h separately. 10 μM of DBCO-Cy5 was added, the cells were incubated in a 37° C. incubator for 30 min, and washed 3 times with PBS; the cells were added with 10 μM of a DiO staining solution, incubated in a 37° C. incubator for 30 min, and washed 3 times with PBS; the cells were fixated with 4% paraformaldehyde at room temperature for 15 min, stained with Hoechst33258 for 15 min, washed 3 times with PBS, and then finally observed under a confocal microscope. A in FIG. 5 reflected the successful metabolic modification of the azido group, and B in FIG. 5 reflected the main localization of the azido group in the cell membrane. Since the envelope of the lentivirus was chemically consistent with the cell membrane of the 293T cells, it was believed that the surface envelope of the lentivirus packaged by Na-293T could be modified to obtain the N₃-LVs.

d. Western Blotting Analysis
(1) Subcellular localization of azido groups analyzed by Western blotting analysis: the 293T cells were pre-inoculated in a T25 culture flask; after a cell fusion degree reached approximately 70%, 50 μM each of Ac4ManNAZ, Ac4GlaNAZ, and Ac4GlcNAZ solutions were added to the cells separately, and incubated in a 37° C. incubator for 48 h; the cytoplasmic proteins, membrane proteins, nuclear proteins, and cytoskeletal proteins were extracted using a subcellular structure protein isolation kit. The cells of each component were quantified by protein extraction and BCA method. The protein was quantified to 5 mg/mL, and then 20 μL of the protein was reacted with 2 μL of 10 μM dibenzocyclooctyne-biotin (DBCO-biotin) at room temperature for 12 h, and then an SDS loading buffer was added to allow boiling at 95° C. for 10 min; a resulting product was subjected to electrophoresis and transfer by a PVDF membrane with 10% SDS-PAGE (100 V, 90 min), and blocked with 5% skim milk for 1 h at room temperature; the PVDF membrane was washed 3 times with TBST, incubated with streptavidin-horseradish peroxidase (Streptavidin-HRP) in TBST for 12 h at 4° C., and washed 3 times with TBST; the PVDF membrane was exposed to an ECL luminescent solution, and images were collected through a gel imaging system.
(2) Time dependence and dose dependence of azidation analyzed by Western blotting analysis: the 293T cells were pre-inoculated in a T25 culture flask; after a cell fusion degree reached approximately 70%, 50 μM of Ac4ManNAZ was added to the cells, and incubated in a 37° C. incubator for 0 h, 6 h, 12 h, 24 h, 48 h and 72 h separately; the cells of each component were subjected to protein extraction, BCA quantification, coupling to DBCO-biotin, denaturation, electrophoresis, membrane transfer, incubation with Streptavidin-HRP, and exposure. The 293T cells were pre-inoculated in the T25 culture flask; after the cell fusion degree reached approximately 70%, 50 μM of Ac4ManNAZ was added to the cells, and incubated in a 37° C. incubator for 48 h, a drug-containing medium was replaced with an ordinary DMEM medium, and the cells in each component were collected at 0 h, 24 h, 48 h, and 72 h separately at this time. The cells of each component were subjected to protein extraction, BCA quantification, coupling to DBCO-biotin, denaturation, electrophoresis, membrane transfer, incubation with Streptavidin-HRP, and exposure. The 293T cells were pre-inoculated in the T25 culture flask; after the cell fusion degree reached approximately 70%, Ac4ManNAZ was added to the cells at gradient concentrations of 0 μM, 1 μM, 5 μM, 10 μM, 25 μM, 50 μM, and 100 μM, and incubated in a 37° C. incubator for 48 h. The cells of each component were subjected to protein extraction, BCA quantification, coupling to DBCO-biotin, denaturation, electrophoresis, membrane transfer, incubation with Streptavidin-HRP, and exposure.
(3) Azidation blocking test: the cells were grouped as follows: a blocking group, an experimental group, and a blank control group. 293T cells were pre-inoculated into a 6-well plate, with 1*10⁵ cells in each well, and then allowed to adhere well overnight. 50 μM of Ac4ManNAZ was added to the cells, and incubated in a 37° C. incubator for 48 h; in the blocking group, 100 μM of DBCO-NH₂ was added to each well, and incubated in a 37° C. incubator for 2 h, while an equal amount of PBS was added to the experimental group and the blank control group separately. A supernatant was discarded, and the cells were washed 3 times with PBS, added with 10 μM of DBCO-Cy5, incubated in a 37° C. incubator for 30 min, washed 3 times with PBS, fixated with 4% paraformaldehyde for 15 min, and tested on a flow cytometer.

### 2. Packaging and Characterization of Azidated Lentivirus (N₃-LVs)

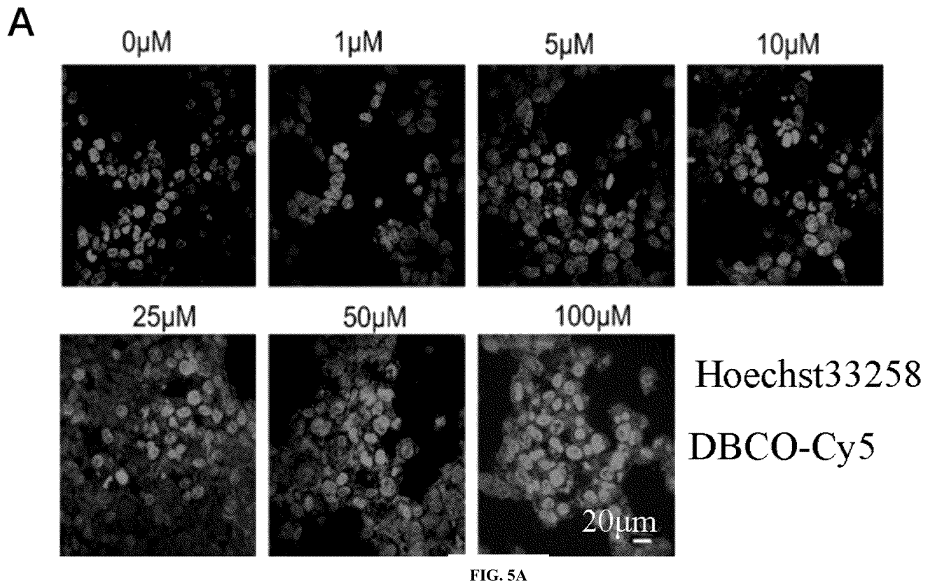

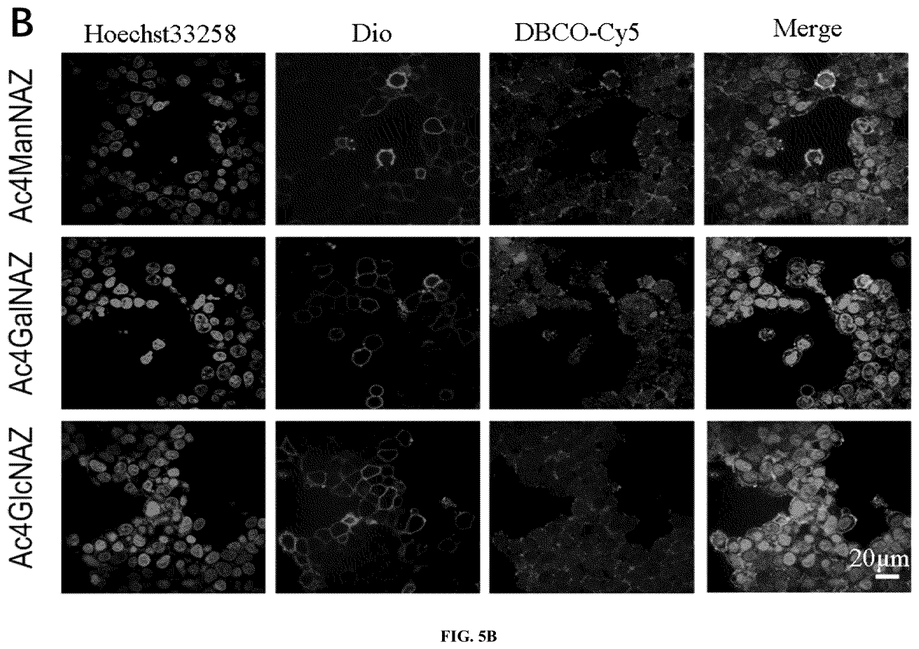

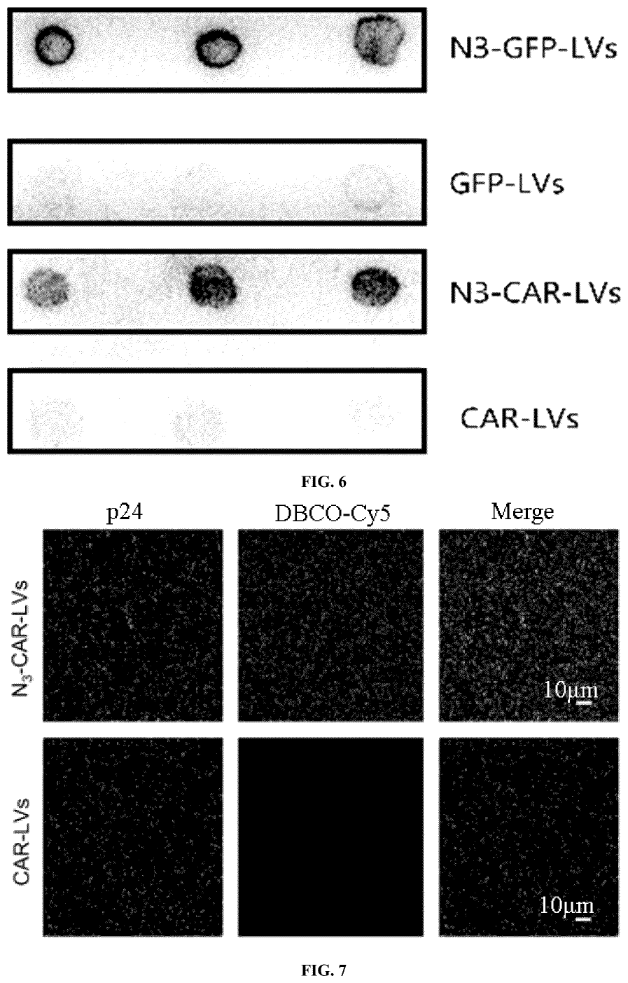

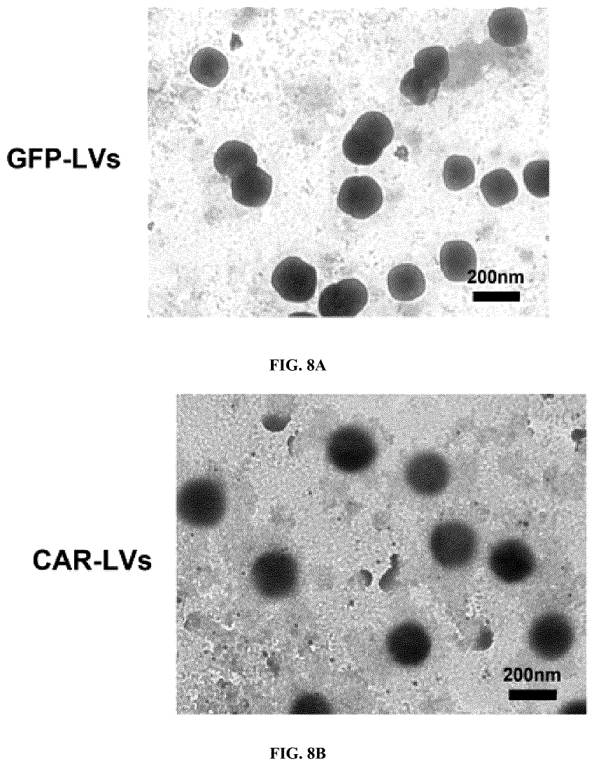

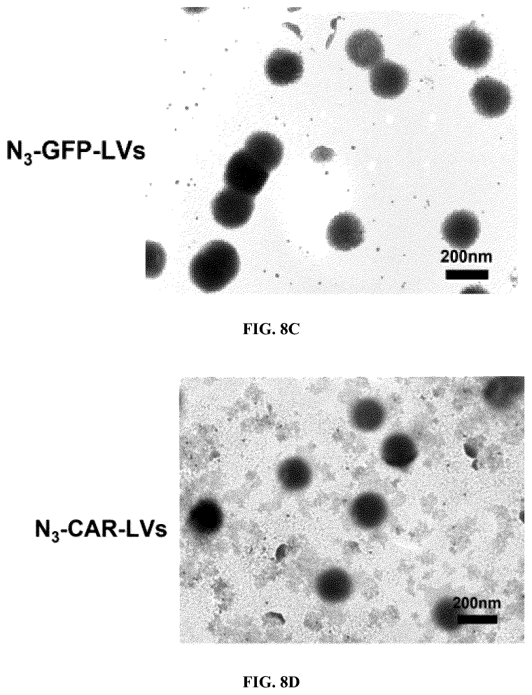

a. Packaging of N₃-LVs
(1) Cell preparation: before transfection, the 293T cells were transferred to a 100 mm culture dish, incubated with 50 μM of Ac4ManNAZ in a 37° C. incubator for 48 h, and then subcultured in due course; where the cells should preferably reach a fusion degree of 80% to 90% before the transfection. Before the transfection, the cell medium was discarded and replaced with a serum-free DMEM, and the cells were starved for 1 h.
(2) Preparation of a transfection complex the next day: for a 10 cm culture dish, a solution A was prepared: a total of 18 μg of plasmid, and vector plasmid: PsPAX2: PMD2.G=9:6:3 were added into 250 μL of Opti-MEM; a solution B was prepared: 45 μL of PEI (1 μg/μL) was dissolved in 250 μL of the Opti-MEM, and mixed well by pipetting. The solutions A and B were each allowed to stand at room temperature for 5 min. The solution B was added to the solution A dropwise, and allowed to stand for another 20 min. 500 μL of a resulting mixture was added to the culture dish dropwise. After 8 h, the medium was replaced with a DMEM complete medium containing 10% fetal bovine serum (where Ac4ManNAZ had a concentration of 50 μM).
(3) Collection of viruses: 48 h and 72 h after the transfection, a cell culture supernatant was aspirated, centrifuged at 2,000 rpm*15 min at 4° C., the cell debris was discarded, and filtered with a 0.22 μm filter; an obtained filtrate was added to an ultrafiltration tube (30 kDa) and centrifuged at 3,300 rpm*25 min, and a virus concentrate was collected and cryopreserved at −80° C.
   b. Characterization of N₃-LVs
(1) TEM observation of appearance: LVs and N₃-LVs were separately added onto a copper grid dropwise, dried at room temperature, added with a drop of phosphotungstic acid (W/V 2%) to allow negative staining for 30 s, dried and observed through TEM, as shown in FIG. 8.
(2) Dot immunoblotting assay (DIBA): spotting: 1 μL of the virus concentrate was spotted on a nitrocellulose membrane, labeled, and dried in a 37° C. incubator for 30 min; blocking: the blocking was conducted with a 5% skimmed milk powder in a shaker at room temperature for 1 h; membrane washing: the membrane was washed with PBST on a shaker for 5 min three times, and finally blot-dried with filter paper; DBCO-biotin addition: 2 μL of 10 μM DBCO-biotin was added to 40 μL of virus solution, and shaken to allow reaction at room temperature for 12 h; the membrane washing was repeated; Streptavidin-HRP addition: the NC membrane and Streptavidin-HRP were co-incubated in TBST for 12 h at 4° C., the membrane was washed 3 times with PBST, and finally added with an ECL luminescent solution to allow exposure, and images were collected through a gel imaging system. The results were shown in FIG. 6.

### 3. Preparation and Characterization of Fluorescently Labeled Lentivirus (Cy5-LVs)

a. Preparation of Cy5-LVs: N₃-LVs and sufficient DBCO-Cy5 (CAS number: 1564286-24-3) were mixed well by pipetting, and incubated in a 37° C. incubator for 30 min; a resulting reaction solution was transferred to an ultrafiltration tube (30 kDa), centrifuged at 3,300 rpm*30 min, excess free DBCO-Cy5 was discarded, and a virus concentrate was collected as the Cy5-LVs.
b. TEM observation of appearance: Cy5-LVs was added onto a copper grid dropwise, dried at room temperature, added with a drop of phosphotungstic acid (W/V 2%) to allow negative staining for 30 s, dried and observed through TEM.
c. Confocal laser observation of Cy5-LVs:
(1) Antibody coating: an anti-p24 antibody diluted 1:500 was added dropwise onto an anti-detachment glass slide, incubated at 4° C. overnight, and then washed 3 times with PBS.
(2) Sample addition: the Cy5-LVs was added dropwise onto the glass slide, incubated at room temperature for 1 h, and fixated in an oven at 42° C. for 15 min.
(3) Blocking: BSA (W/V 2%) was added dropwise to the glass slide and incubated at room temperature for 1 h.
(4) Addition of secondary antibody: a goat anti-mouse secondary antibody (FITC-labeled) was added dropwise to the glass slide, incubated at room temperature for 1 h, and washed 3 times with PBS.
(5) Sealing: a drop of sealing agent was added onto the glass slide and covered with a coverslip to prevent bubbles from being generated and sliding.
(4) Image collection: the glass slide was observed through a laser confocal microscope. As shown in FIG. 7, lentivirus surface marker p24 was a specific molecule and could be used as a marker, and DBCO-Cy5 was used as an Na group detection probe. The co-localization of the above two proved that the fluorescent labeling of lentivirus was successful. This group was easy and efficient to modify, and could be coupled with the fluorescent probe DBCO-Cy5 through a click reaction under mild conditions without any catalyst.

### 4. Dynamic Monitoring of Lentivirus Infection on Host Cells Based on Cy5-LVs

293T cells were pre-inoculated into a confocal dish, with 1*10⁵ cells in each well, and then allowed to adhere well overnight. The next day, the original medium was replaced with 2 mL of a fresh medium containing 6 μg/mL polybrene, and an appropriate amount of virus suspension was added. The Cy5-LVs was added at a certain MOI and incubated at 37° C. The medium was discarded at predetermined time points of 0.5 h, 1 h, 2 h, 4 h, 8 h, 12 h, 24 h, 48 h, and 72 h, and the cells were stained with a cell membrane probe DiO, a lysosome probe Lyso-Tracker, an endoplasmic reticulum fluorescent probe ER-Tracker, and a nuclear probe Hoechst33342. The cells were fixated with 4% paraformaldehyde for 15 min, and the subcellular localization of Cy5-LVs in 293T cells was observed by a laser confocal microscope or super-resolution fluorescence microscope to monitor the dynamic infection behavior.

Analysis: The lentivirus is covered with an envelope on its surface and is an enveloped virus. During the packaging of lentivirus, when the virus budded, it took away the cell membrane components of 293T cells and formed its own envelope. Therefore, the virus envelope and the 293T cell membrane showed the same chemical essence. The cell membrane is mainly composed of a phospholipid bilayer and surface glycoproteins, and contains a large number of sugar groups. By adding azidation-modified non-natural sugars (azido sugars) to the 293T cell medium, azidation of the cell membrane system could be achieved through intrinsic sugar metabolism, namely bioorthogonal reaction. Accordingly, the envelope surface of lentivirus produced through N₃-293T packaging could also express the azido group (N₃-LVs). Based on the cellular sugar metabolism mechanism, modification of cell membrane glycoproteins with small-molecular reporter groups is a reliable technical means to achieve envelope labeling of lentiviruses. The chemical group of this glycochemistry-mediated modification had a small molecular weight. Moreover, the modification was a natural metabolic synthesis process, which did not affect the normal physiological metabolism and genome function of 293T cells, nor did it affect the transfection activity of the virus. As a result, this method was non-invasive for covalent modification of viruses, and N₃-LVs had the same infection function as that of unmodified LVs and could mediate the stable expression of target genes.

It will be clear to those skilled in the art that various modifications to the above examples can be made without departing from the general spirit and concept of the present disclosure. These modifications shall all fall within the protection scope of the present disclosure. The claimed protection schemes of the present disclosure shall be determined by the claims.

1. A preparation method of a fluorescently labeled lentiviral vector, comprising the following steps:
preparation of N₃-LVs: subjecting a 293T cell and an azido sugar to co-incubation, such that a cell membrane system is azidated through sugar metabolism to obtain an N₃-293T cell capable of expressing an azido group on an envelope surface, and subjecting the N₃-293T cell and a plasmid packaging system to co-transfection to form a lentiviral vector N₃-LVs; and
preparation of Cy5-LVs: mixing the N₃-LVs with a sufficient amount of an azide dibenzocyclooctyne-cyanine 5 (DBCO-Cy5) to allow co-incubation, removing excess DBCO-Cy5, and collecting a virus concentrate to obtain a fluorescently labeled lentiviral vector Cy5-LVs.

2. The preparation method of a fluorescently labeled lentiviral vector according to claim 1, wherein the plasmid packaging system is selected from the group consisting of a three-plasmid packaging system and a four-plasmid packaging system;
the three-plasmid packaging system comprises: a vector plasmid, and packaging plasmids PsPAX2, PMD2.G; and
the four-plasmid packaging system comprises: the vector plasmid, and packaging plasmids pVSV-G, pGap/pol, and pRev.

3. The preparation method of a fluorescently labeled lentiviral vector according to claim 2, wherein the N₃-LVs is prepared specifically according to the following steps:
subjecting the 293T cell and the azido sugar to the co-incubation and subculture in a cell medium, discarding the cell medium, and transferring a resulting culture product into a serum-free medium; and
adding a mixture of a solution A and a solution B dropwise into the serum-free medium, replacing the serum-free medium with a Dulbecco's modified eagle medium (DMEM) complete medium containing fetal bovine serum, conducting transfection, aspirating a resulting cell culture supernatant to obtain a virus concentrate, thereby obtaining the N₃-LVs; and
the solution A is prepared by placing the vector plasmid, the PsPAX2, and the PMD2.G in a serum-reduced medium, and the solution B is prepared by dissolving polyethylenimine (PEI) in the serum-reduced medium; alternatively,
the solution A is prepared by placing the vector plasmid, the pVSV-G, the pGap/pol, and the pRev in the serum-reduced medium, and the solution B is prepared by dissolving the PEI in the serum-reduced medium.

4. The preparation method of a fluorescently labeled lentiviral vector according to claim 3, wherein the vector plasmid, the PsPAX2, and the PMD2.G in the solution A are at a ratio of 3:2:1 in parts by weight; alternatively, the vector plasmid, the pVSV-G, the pGap/pol, and the pRev in the solution A are at a ratio of 10.5:1.5:5:3 in parts by weight; and the solution B has the PEI at a concentration of 1 μg/μL.

5. The preparation method of a fluorescently labeled lentiviral vector according to claim 3, wherein the 293T cell and the azido sugar subjected to the co-incubation and the subculture in the cell medium have a fusion degree of 80% to 90%.

6. The preparation method of a fluorescently labeled lentiviral vector according to claim 1, wherein the azido sugar is any one selected from the group consisting of N-azidoacetylmannosamine-tetraacylated (Ac4ManNAZ), N-azidoacetylgalactosamine-tetraacylated (Ac4GlaNAZ), and N-azidoacetylglucosamine-tetraacylated (Ac4GlcNAZ); and
the 293T cell and the azido sugar are subjected to the co-incubation in the cell medium at 35° C. to 38° C.; and the N₃-LVs and a sufficient amount of the azide DBCO-Cy5 serving as a reaction probe are subjected to the co-incubation at 35° C. to 38° C.

7. The preparation method of a fluorescently labeled lentiviral vector according to claim 2, wherein the azido sugar is any one selected from the group consisting of N-azidoacetylmannosamine-tetraacylated (Ac4ManNAZ), N-azidoacetylgalactosamine-tetraacylated (Ac4GlaNAZ), and N-azidoacetylglucosamine-tetraacylated (Ac4GlcNAZ); and the 293T cell and the azido sugar are subjected to the co-incubation in the cell medium at 35° C. to 38° C.; and the N₃-LVs and a sufficient amount of the azide DBCO-Cy5 serving as a reaction probe are subjected to the co-incubation at 35° C. to 38° C.

8-10. (canceled)

11. The preparation method of a fluorescently labeled lentiviral vector according to claim 3, wherein the azido sugar is any one selected from the group consisting of N-azidoacetylmannosamine-tetraacylated (Ac4ManNAZ), N-azidoacetylgalactosamine-tetraacylated (Ac4GlaNAZ), and N-azidoacetylglucosamine-tetraacylated (Ac4GlcNAZ); the 293T cell and the azido sugar are subjected to the co-incubation in the cell medium at 35° C. to 38° C.; and the N₃-LVs and a sufficient amount of the azide DBCO-Cy5 serving as a reaction probe are subjected to the co-incubation at 35° C. to 38° C.

12. The preparation method of a fluorescently labeled lentiviral vector according to claim 4, wherein the azido sugar is any one selected from the group consisting of N-azidoacetylmannosamine-tetraacylated (Ac4ManNAZ), N-azidoacetylgalactosamine-tetraacylated (Ac4GlaNAZ), and N-azidoacetylglucosamine-tetraacylated (Ac4GlcNAZ); the 293T cell and the azido sugar are subjected to the co-incubation in the cell medium at 35° C. to 38° C.; and the N₃-LVs and a sufficient amount of the azide DBCO-Cy5 serving as a reaction probe are subjected to the co-incubation at 35° C. to 38° C.

13. The preparation method of a fluorescently labeled lentiviral vector according to claim 5, wherein the azido sugar is any one selected from the group consisting of N-azidoacetylmannosamine-tetraacylated (Ac4ManNAZ), N-azidoacetylgalactosamine-tetraacylated (Ac4GlaNAZ), and N-azidoacetylglucosamine-tetraacylated (Ac4GlcNAZ);
the 293T cell and the azido sugar are subjected to the co-incubation in the cell medium at 35° C. to 38° C.; and the N₃-LVs and a sufficient amount of the azide DBCO-Cy5 serving as a reaction probe are subjected to the co-incubation at 35° C. to 38° C.

14. The preparation method of a fluorescently labeled lentiviral vector according to claim 7, wherein the azido sugar has a concentration of 50 μM.

15. The preparation method of a fluorescently labeled lentiviral vector according to claim 11, wherein the azido sugar has a concentration of 50 μM.

16. The preparation method of a fluorescently labeled lentiviral vector according to claim 12, wherein the azido sugar has a concentration of 50 μM.

17. The preparation method of a fluorescently labeled lentiviral vector according to claim 13, wherein the azido sugar has a concentration of 50 μM.

18. A fluorescently labeled lentiviral vector N₃-LVs prepared by the preparation method of a fluorescently labeled lentiviral vector according to claim 1.

19. The fluorescently labeled lentiviral vector N₃-LVs according to claim 18, wherein:
the plasmid packaging system is selected from the group consisting of a three-plasmid packaging system and a four-plasmid packaging system;
the three-plasmid packaging system comprises: a vector plasmid, and packaging plasmids PsPAX2, PMD2.G; and
the four-plasmid packaging system comprises: the vector plasmid, and packaging plasmids pVSV-G, pGap/pol, and pRev.

20. The fluorescently labeled lentiviral vector N₃-LVs according to claim 19, wherein the N₃-LVs is prepared specifically according to the following steps:
subjecting the 293T cell and the azido sugar to the co-incubation and subculture in a cell medium, discarding the cell medium, and transferring a resulting culture product into a serum-free medium; and
adding a mixture of a solution A and a solution B dropwise into the serum-free medium, replacing the serum-free medium with a Dulbecco's modified eagle medium (DMEM) complete medium containing fetal bovine serum, conducting transfection, aspirating a resulting cell culture supernatant to obtain a virus concentrate, thereby obtaining the N₃-LVs; and
the solution A is prepared by placing the vector plasmid, the PsPAX2, and the PMD2.G in a serum-reduced medium, and the solution B is prepared by dissolving polyethylenimine (PEI) in the serum-reduced medium; alternatively,
the solution A is prepared by placing the vector plasmid, the pVSV-G, the pGap/pol, and the pRev in the serum-reduced medium, and the solution B is prepared by dissolving the PEI in the serum-reduced medium.

21. The fluorescently labeled lentiviral vector N₃-LVs according to claim 20, wherein the vector plasmid, the PsPAX2, and the PMD2.G in the solution A are at a ratio of 3:2:1 in parts by weight; alternatively, the vector plasmid, the pVSV-G, the pGap/pol, and the pRev in the solution A are at a ratio of 10.5:1.5:5:3 in parts by weight; and the solution B has the PEI at a concentration of 1 μg/μL.

22. The fluorescently labeled lentiviral vector N₃-LVs according to claim 20, wherein the 293T cell and the azido sugar subjected to the co-incubation and the subculture in the cell medium have a fusion degree of 80% to 90%.

23. The preparation method of a fluorescently labeled lentiviral vector according to claim 6, wherein the azido sugar has a concentration of 50 μM.
